(19) United States
(12) Patent Application Publication
(10) Pub. No.: US 2010/0266656 A1
(43) Pub. Date: Oct. 21, 2010

(54) Splittable Elastomeric Drug Delivery Device

(75) Inventor: David Johnson, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/425,577

(22) Filed: Apr. 17, 2009

(51) Int. Cl. A61M 25/10; B05D 3/00

(52) U.S. Cl. 424/423; 604/509; 604/103.01; 427/2.25

(57) ABSTRACT

A system for treating a vascular condition including a catheter and a splittable elastomeric drug delivery device. The splittable elastomeric drug delivery device includes a balloon disposed on the catheter. The balloon includes a first elastic layer and a second elastic layer. A therapeutic agent layer is disposed on at least a portion of the first elastic layer, and the second elastic layer is disposed on the first elastic layer and the therapeutic agent layer. The first elastic layer has a first elongation-at-break percentage and the second elastic layer has a second elongation-at-break percentage.

### TECHNICAL FIELD

The technical field of this disclosure relates to vascular treatment devices. More specifically, the disclosure relates to a splittable elastomeric drug delivery device.

### BACKGROUND OF THE INVENTION

Heart disease, specifically coronary artery disease, is a major cause of death, disability, and healthcare expense in the United States and other industrialized countries. A number of methods and devices for treating coronary artery disease have been developed, some of which are specifically designed to treat the complications resulting from atherosclerosis and other forms of coronary arterial narrowing.

One method for treating such vascular conditions is percutaneous transluminal coronary angioplasty (PTCA). During PTCA, a balloon catheter device is inflated to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter improves blood flow.

However, soon after the procedure, a significant proportion of treated vessels restenose. Various methods have been developed to prevent or inhibit this restenosis. One method is to provide a drug or therapeutic agent to assist in preventing inflammation, infection, thrombosis, and proliferation of cell growth that can occlude the vessel lumen.

There are several drawbacks to current drug eluting technology. One of the drawbacks is that the drug can be exposed to the environment before deployment to the treatment site. Handling the drug before delivery can cause the drug to lose some of its efficacy. Another problem is that the drug is sometimes accidentally released before reaching the treatment site. Yet another problem is that many times the drug delivery device must be cured at high temperatures in order to achieve the desired properties of the drug delivery device. However, curing at high temperatures after the drug has been added to the drug delivery device can damage the drug and reduce its therapeutic effectiveness. Therefore, it would be desirable to have a system and method for treating a vascular condition that can overcome the aforementioned and other disadvantages.

### SUMMARY OF THE INVENTION

One aspect of the present invention provides a system for treating a vascular condition. The system includes a catheter and a splittable elastomeric drug delivery device. The splittable elastomeric drug delivery device includes a balloon disposed on the catheter. The balloon includes a first elastic layer and a second elastic layer. A therapeutic agent layer is disposed on at least a portion of the first elastic layer, and the second elastic layer is disposed on the first elastic layer and the therapeutic agent layer. The first elastic layer has a first elongation-at-break percentage and the second elastic layer has a second elongation-at-break percentage.

Another aspect of the present invention provides a method of formation of a splittable elastomeric drug delivery device. The method includes forming a first coat on a balloon mandrel; curing the first coat to form a first elastic layer; forming a therapeutic agent layer disposed on at least a portion of the first elastic layer; forming a second coat disposed on the first elastic layer and the therapeutic agent layer; and curing the second coat to form a second elastic layer. The first elastic layer has a first elongation-at-break percentage and the second elastic layer has a second elongation-at-break percentage.

Another aspect of the present invention provides a method for treating a vascular condition. The method includes advancing a splittable elastomeric drug delivery device to a treatment site and inflating the balloon. The device includes a balloon having a first elastic layer, a therapeutic agent layer disposed over at least a portion of the first elastic layer, and a second elastic layer disposed over the first elastic layer and the therapeutic agent layer. The inflating splits the second elastic layer to expose the therapeutic agent layer to the treatment site.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

### BRIEF DESCRIPTION OF THE DRAWINGS

FIG. 1A is a perspective view of a system for treating a vascular condition in accordance with the present invention.

FIG. 1B is a cross section view of a system for treating a vascular condition in accordance with the present invention.

FIG. 2 is a flow diagram for a method of manufacturing an elastomeric drug delivery device for treating a vascular condition in accordance with the present invention.

FIG. 3A is a side view of a splittable elastomeric drug delivery device in a deflated delivery state in accordance with the present invention.

FIG. 3B is a side view of a splittable elastomeric drug delivery device in an expanded therapeutic state in accordance with the present invention.

FIG. 4 is a flow diagram for a method of using an elastomeric drug delivery device for treating a vascular condition in accordance with the present invention.

FIGS. 5A-5G are detailed cross section views of another embodiment of the elastomeric drug delivery device in accordance with the present invention.

FIG. 6 is a flow diagram for a method of manufacturing another embodiment of an elastomeric drug delivery device for treating a vascular condition in accordance with the present invention.

### DETAILED DESCRIPTION

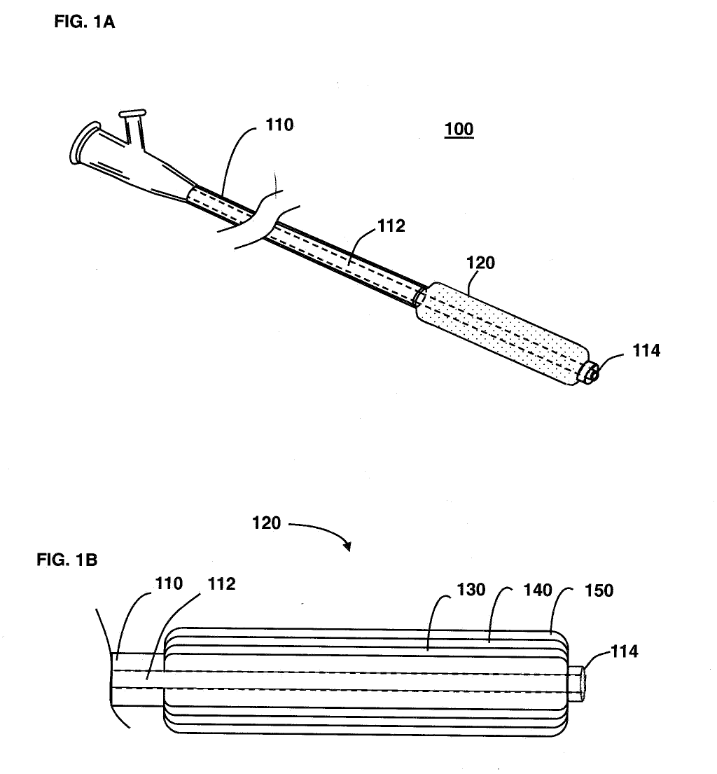

FIG. 1A is a perspective view of a system 100 for treating a vascular condition in accordance with the present invention and FIG. 1B is a cross section view of the balloon of the system 100. The system 100 includes a catheter 110 and a balloon 120 disposed on the catheter 110 toward the distal tip 114 of the catheter 110. The balloon 120 includes a first elastic layer 130, a therapeutic agent layer 140 disposed on the first elastic layer 130, and a second elastic layer 150 disposed on the first elastic layer 130 and the therapeutic agent layer 140.

The catheter 110 includes an inflation lumen 112 for inflating the drug delivery balloon 120. The catheter 110 may be any catheter known in the art for delivering a drug delivery balloon to a treatment site within a vessel. The catheter 110 may be a percutaneous transluminal coronary angioplasty (PTCA) balloon catheter. Methods for the formation of the first elastic layer 130, the therapeutic agent layer 140, and the second elastic layer 150 are discussed in detail below.

Referring to FIG. 1B, in one embodiment the first elastic layer 130 is a high temperature vulcanized silicone dispersion. The resulting material is a high elongation-at-break material with an elongation-at-break percentage of at least 1000%.

The therapeutic agent layer 140 is disposed on at least a portion of the first elastic layer 130 by, for example, dipping or spraying. The therapeutic agent layer 140 may include, for example, an antineoplastic agent, an antiproliferative agent, an antibiotic, an antithrombogenic agent, an anticoagulant, an antiplatelet agent, an anti-inflammatory agent, or combinations of the above. Various other therapeutic agents such as fibrinolytics, therapeutic proteins or peptides, recombinant DNA products, or other bioactive agents, diagnostic agents, radioactive isotopes, or radiopaque substances may be included in the therapeutic agent layer 140 depending on the anticipated needs of the patient. Those of skill in the art will appreciate that the therapeutic agent in the therapeutic agent layer 140 may be included in any form allowing the therapeutic agent to flow through the split formed in the second elastic layer, such as a liquid, a loose powder, a paste, a capsule, or the like. The therapeutic agent may exit the split in its original form, or may be mixed with or dissolved in fluid flowing in the lumen in which the balloon 120 is deployed. In one embodiment, the therapeutic agent layer 140 can be a powdered drug, which is defined herein as a ground, pulverized, or otherwise finely dispersed solid particles of a therapeutic agent, and can include encapsulated particles or nano-particles. In another embodiment, the therapeutic agent layer 140 can be a low temperature drug, which is defined herein as a therapeutic agent that is damaged by exposure to temperatures other than low temperatures, such as by exposure to curing at elevated temperatures. The formulation containing the therapeutic agent layer 140 may additionally contain excipients including solvents or other solubilizers, stabilizers, suspending agents, antioxidants, and preservatives, as needed to deliver an effective dose of the therapeutic agent to the treatment site.

The second elastic layer 150 is disposed on the first elastic layer 130 and the therapeutic agent layer 140. In one embodiment, the second elastic layer 150 is an oxime cured silicone dispersion. The resulting material is a lower elongation-at-break material than the first elastic layer, preferably with an elongation-at-break percentage in the range of 550% to 750%.

Those skilled in the art will appreciate that the first elastic layer 130 and the second elastic layer 150 may be formed from any biocompatible polymeric material having elastomeric characteristics such as those described above. The elastomeric material may be, for example, high temperature vulcanized or room temperature vulcanized silicones, or combinations thereof. In one embodiment, the elongation-at-break percentage differential between the first elastic layer and the second elastic layer is at least 250%.

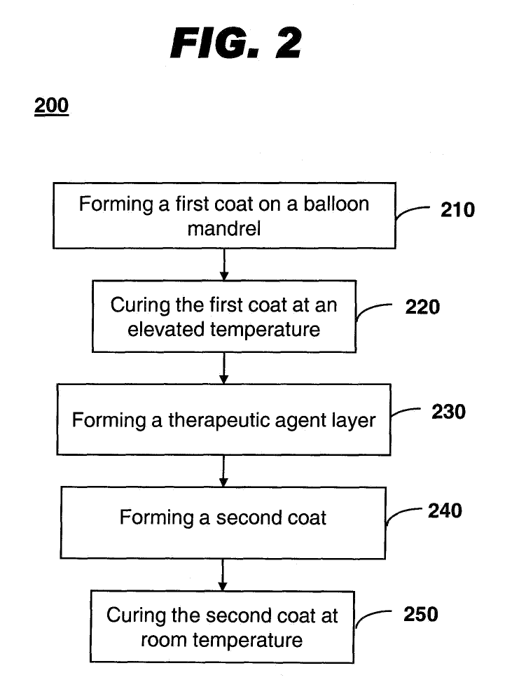

FIG. 2 is a flow diagram for a method 200 of manufacturing an elastomeric drug delivery device for treating a vascular condition. The method 200 includes forming a first coat on a balloon mandrel 210; curing the first coat to form a first elastic layer 220; forming a therapeutic agent layer disposed on the first coat 230; forming a second coat disposed on the first coat and the therapeutic agent layer 240; and curing the second coat to form a second elastic layer 250.

Forming the first coat on the balloon mandrel 210 can be accomplished by any method known in the art such as dipping, spraying, painting, wiping, rolling, printing and combinations thereof. The mandrel is a mold having an outer surface which yields the desired dimensions and shape of the elastomeric drug delivery device. In one embodiment, the mandrel is a mold having the dimensions and shape required to form a spherical elastomeric drug delivery device.

The first coat is formed on the mandrel by dipping the mandrel in a liquid undercoat medium that contains an elastomeric polymer. The liquid undercoat medium may be latex or a solution of the polymer in an organic solvent. Organic solvents may be, for example, ethers, amines, esters or alcohols. In one embodiment, the liquid undercoat medium is a solution of silicone in xylene. In another embodiment, the undercoat medium is a solution of silicone in hexane. Dipping the mandrel into the liquid undercoat medium and then withdrawing the mandrel will leave a film of the liquid undercoat medium over an outer surface of the mandrel. The thickness of the first coat may be increased by dipping the mandrel multiple times in order to produce a first coat of a desired thickness.

The undercoat elastomeric film can be partially cured on the mandrel between each dipping to allow for the adhesion of the first intermediate layers to increase the thickness of the first coat. The length of time that the dipped mandrel is cured between dips to provide sufficient adhesion depends on such factors as type of polymer, type of solvent and the desired degree of viscosity. Once the desired thickness is achieved, the first coat may be cured 220, preferably at an elevated temperature, for example from about 250° F. to about 350° F. for about 120 minutes to about 150 minutes to form the first elastic layer. In one embodiment, the cure is a platinum cure carried out at about 170° F. for approximately 45 minutes, followed by an additional approximate 135 minutes at about 300° F. Those of skill in the art will appreciate that the cure may be carried out at different combinations of time and temperature for the same effect. For example, the cure may be carried out at lower temperatures for a longer period of time.

Next, forming the therapeutic agent layer 230 includes applying a therapeutic agent to the entire first elastic layer or any portion thereof. A portion of the first coat may be masked before dipping the mandrel into the overcoat solution in order to suit a particular application. The therapeutic agent layer may be applied by any method known in the art such as, for example, by dipping, spraying, painting, wiping, rolling, printing, and combinations thereof. The therapeutic agent layer can be preferentially applied to the apex of the balloon, i.e., to the portion of the balloon that becomes an apex portion of the balloon upon inflation where the circumference of the balloon changes dramatically. In one embodiment, the elastomeric drug delivery device is secured to a delivery catheter prior to adding the therapeutic agent layer.

After forming the therapeutic agent layer 230, the second coat is formed disposed on the first layer and the therapeutic agent layer 240. The mandrel with the therapeutic coat is dipped into an overcoat polymeric solution to form the second coat. The forming of the second coat 240 is similar to the forming of the first coat. The second coat can also be formed by any method known in the art such as dipping, spraying, painting, wiping, rolling, printing and combinations thereof. Application of the second coat traps the therapeutic agent coat between the first coat and the second coat. The thickness of the second coat may be increased by performing additional dipping and drying cycles to form second intermediate layers. The second intermediate layers can be added until the desired thickness of the second coat is achieved. The thickness of the second coat may vary in order to make certain portions of the second coat more elastic than other portions. Once the desired thickness is achieved, the second coat may be cured 250, for example at room temperatures ranging from about 70° F. to 77° F., preferably for at least 24 hours, to form the second elastic layer.

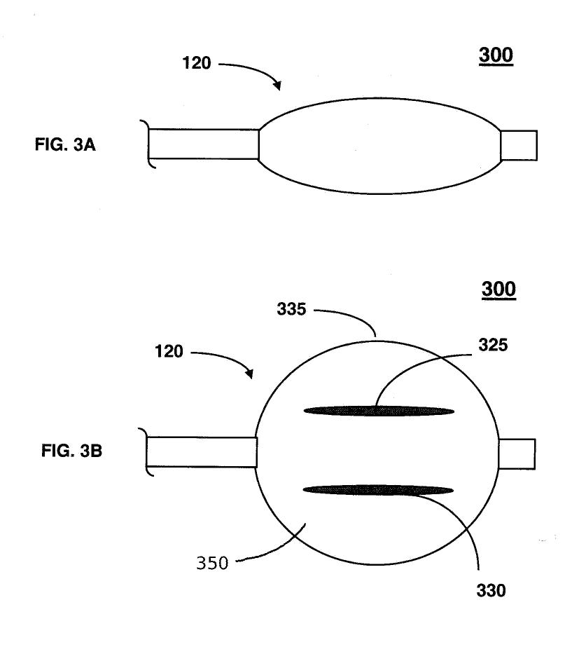

FIG. 3A is a side view of a splittable elastomeric drug delivery device with the balloon 120 in a deflated delivery state. The therapeutic agent layer is trapped between the first coat and the second coat (not shown).

FIG. 3B is a side view of a splittable elastomeric drug delivery device with the balloon 120 in an expanded therapeutic state. In the expanded therapeutic state, the inflated drug delivery device causes the second elastic layer 350 to split and expose the therapeutic agent layer 330 through the resulting split 325. Except for the resulting split 325, the second elastic layer 350 remains intact. In one embodiment, one split is generated when the drug delivery device is inflated. In another embodiment, multiple splits are generated when the drug delivery device is inflated. In one embodiment, the expanded therapeutic state 320 that causes the second elastic layer 350 to split is when the body to neck ratio of the inflated balloon is from about a 7:1 ratio to an 8.5:1 ratio. In one exemplary embodiment as illustrated in FIG. 3B, the inflated balloon 120 has a spherical shape, including an apex 335 where the apex 335 is the highest point of the balloon. In one embodiment, the therapeutic agent is concentrated in the proximity of the apex 335 of the spherical shaped balloon 120. Those skilled in the art will appreciate the therapeutic agent can exit the split 325, the fluid in the vessel lumen can enter the split 325, or a combination of the two, as desired for a particular application.

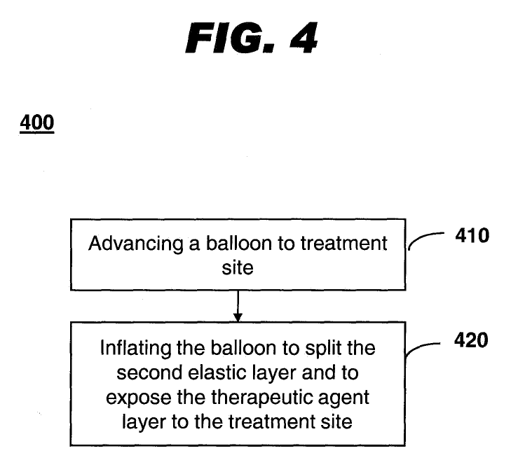

FIG. 4 is a flow diagram for a method 400 of using an elastomeric drug delivery device for treating a vascular condition. The elastomeric delivery device is a balloon which includes a first elastic layer, a therapeutic agent layer, and a second elastic layer. The method 400 includes advancing a balloon to a treatment site 410 and inflating the balloon to split the second elastic layer and to expose the therapeutic agent layer to the treatment site 420.

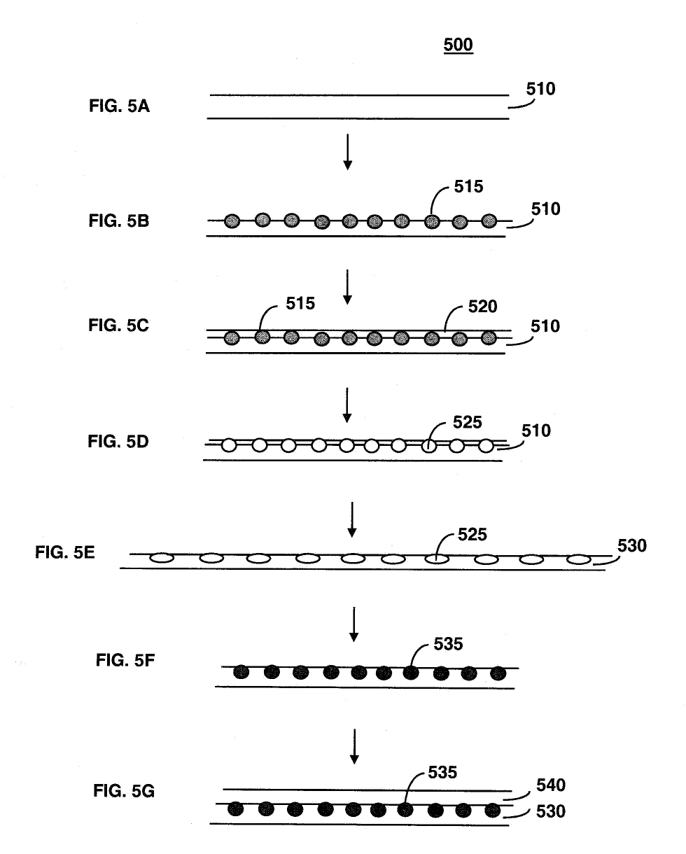

FIGS. 5A-5G are detailed cross section views depicting steps in the manufacture of such an embodiment of the elastomeric drug delivery device in accordance with the present invention. In this embodiment, the therapeutic agent layer is formed by the creation of expandable pores and the filling of the pores with therapeutic agent.

FIG. 5A is a detailed cross section of another embodiment of the first coat 510 of the elastomeric drug delivery device in accordance with the present invention. FIG. 5B is a detailed cross-section of granular particles 515 deposited on the first coat 510. FIG. 5C is a detailed cross section of the first intermediate layers 520 added to the first coat 510. The first intermediate layers 520 trap the granular particles 515 within the first coat 510. FIG. 5D is a detailed cross-section of the first elastic layer 530 after the first coat is cured and the granular particles are dissolved, thereby creating expandable pores 525. Methods for dissolving the granular particles and creating expandable pores are discussed in detail below. FIG. 5E is a detailed cross-section of the expandable pores 525 when the elastomeric drug delivery device is inflated. FIG. 5F is a detailed cross section of the therapeutic agent 535 loaded in the expandable pores. FIG. 5G is a detailed cross section of the therapeutic agent 535 trapped within the expandable pores by the second elastic layer 540.

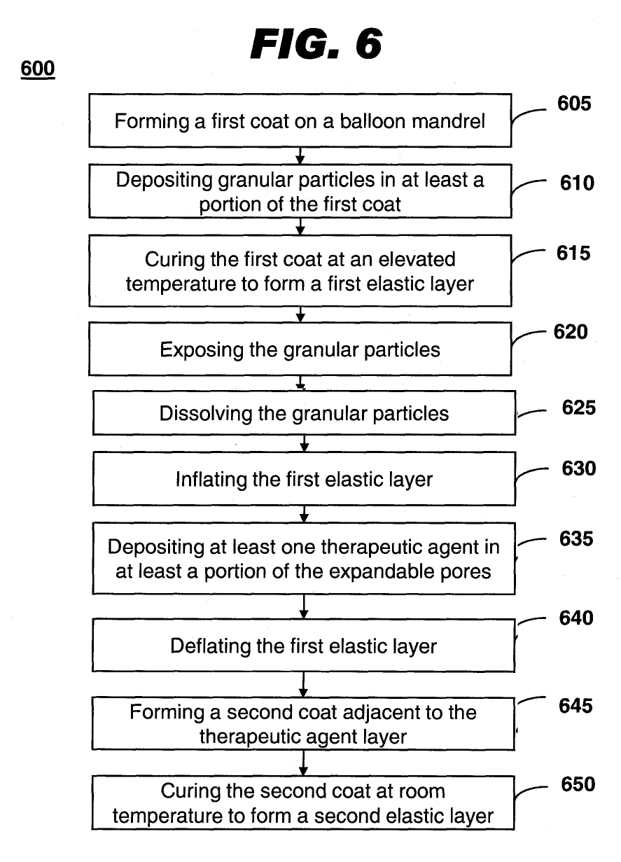

FIG. 6 is a flow diagram for a method of manufacturing the embodiment of FIGS. 5A-5G. The method 600 includes forming a first coat on a balloon mandrel 605; depositing granular particles on at least a portion of the first coat 610; curing the first coat 615; exposing the granular particles 620; dissolving the granular particles 625; inflating the first elastic layer 630; depositing at least one therapeutic agent in at least a portion of the expandable pores to form a therapeutic agent layer 635; deflating the first elastic layer 640; forming a second coat disposed on the first elastic layer and the therapeutic agent layer 645; and curing the second coat to form the second elastic layer 650.

The elastomeric drug delivery device of this embodiment is formed on a mandrel using a dipping process similar to the process described with respect to FIG. 2. Referring to FIG. 6, forming the first coat on the mandrel 605 includes dipping the mandrel into a liquid undercoat medium that contains an elastomeric polymer. To deposit the granular particles on at least a portion of the first coat 610, the mandrel having the first coat is immersed in a fluidized particle bath. The fluidized particle bath is an aerated bath where air or other gas is passed through granular particles to keep the particles mobile. The fluidized particle bath can be a fluidized salt bath that contains crystalline sodium chloride. Though granular salt is preferred, the fluidized particle bath may contain any particle that is soluble in a liquid such as water, as discussed in more detail below. The size and shape of the granular particle contained in the fluidized salt bath determines the size and shape of the expandable pores included in the first elastic layer. In one embodiment, the expandable pores are crystalline shaped pores corresponding to the size and shape of the crystalline structure of the fluidized particles.

When the coated mandrel is dipped into fluidized salt, salt particles adhere to the surface. The amount of granular salt that adheres to the first coat depends on such factors as, for example, the dipping technique, the time of immersion, the amount of air flow through the salt, the size of the granular particles, and the like. The fluidized granular salt particles will adhere to those portions having a first coat. The particles may be disposed on the first coat by other methods, such as, for example, by spraying the particles onto the first coat.

In one embodiment, the mandrel with the first coat including adhered particles is dipped into the undercoat polymeric solution containing the first coat to add first intermediate layers to the first coat. In another embodiment, a different polymeric solution having a higher viscosity than the first coat may be used to add intermediate layers to the first coat. The application of the first intermediate layers traps the granular particles within the first coat. The thickness of the first elastic layer may be increased by performing additional dipping and drying cycles to add more intermediate layers. The coated mandrel is dipped and partially cured until the desired thickness of the first coat is achieved. In one embodiment, the first coat is applied in a series of dips so that the adhered salt particles are substantially covered by the undercoat polymeric solution. Once the desired thickness is achieved, the first coat may be cured 615 to form the first elastic layer. The first coat may be cured at about 250° F. to about 350° F. for about 120 minutes to about 150 minutes. In one embodiment, the first coat is cured at 170° F. for approximately 45 minutes followed by an additional approximate 135 minutes at about 200° F. Those skilled in the art will appreciate that the cure may be carried out at different combinations of time and temperature for the same effect as desired for a particular application.

To form the expandable pores, the first coat can be scrubbed or otherwise brushed to remove a thin layer of the first coat in order to break the surface and to expose the embedded soluble particles 620. Once exposed, the mandrel is placed in a bath containing a liquid such as water to dissolve the particles 625, leaving expandable pores within the first coat. Any loose or poorly adhered particles may be removed after the mandrel is removed from the fluidized particle bath.

The first coat of the elastomeric drug delivery device is then removed from the mandrel. To deposit the therapeutic agent in the expandable pores 640, the elastomeric drug delivery device is inflated 630 to open and expand the pores. Once expanded, the therapeutic agent is deposited in a least a portion of the expanded pores 635. The therapeutic agent may be applied by any method known in the art such as, for example, by dipping, spraying, painting, wiping, rolling, printing, and combinations thereof. In one embodiment, the elastomeric drug delivery device is secured to a delivery catheter prior to loading the therapeutic agent. In another embodiment, the elastomeric drug delivery device is secured to an inflation mandrel, loaded with the therapeutic agent, removed from the inflation mandrel and secured to the delivery catheter. A portion of the first coat can be masked before dipping the mandrel into the second coat solution 645 or prior to applying the therapeutic agent 635. The mask, if present, may be removed after application of the additional layers to the first coat as described above or after exposure of the granular particles 620. Once the therapeutic agent is applied, the elastomeric drug delivery device is deflated 640, thereby collapsing the pore openings to trap the therapeutic agent within the expandable pores. In this embodiment, the embedded therapeutic agent in the pores is the therapeutic agent layer.

Next, the drug delivery device is placed back on the mandrel in order to form the second coat disposed on the first coat and the therapeutic agent layer 645. The mandrel with the first coat and therapeutic agent is then dipped into an overcoat polymeric solution to form the second coat adjacent to the therapeutic agent layer 645. The second coat is applied in a similar manner to the second coat as described for FIG. 2. Referring to FIG. 6, the second coat is then cured 650, preferably at about room temperature for at least 24 hours, to form the second elastic layer.

It is important to note that FIGS. 1-6 illustrate specific applications and embodiments of the present invention, and are not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a vascular condition, the system comprising:
a catheter; and
a splittable elastomeric drug delivery device comprising a balloon disposed on the catheter, the balloon including a first elastic layer; a therapeutic agent layer disposed on at least a portion of the first elastic layer; and a second elastic layer disposed on the first elastic layer and the therapeutic agent layer, wherein the first elastic layer has a first elongation-at-break percentage and the second elastic layer has a second elongation-at-break percentage.

2. The system of claim 1, wherein the first elastic layer is a platinum cure silicone dispersion.

3. The system of claim 1, wherein the first elongation-at-break percentage is at least 1000%.

4. The system of claim 1, wherein the therapeutic agent layer includes expandable pores, the therapeutic agent being disposed within the expandable pores.

5. The system of claim 1, wherein the therapeutic agent layer is a powdered drug.

6. The system of claim 1, wherein the therapeutic agent layer is a low temperature drug.

7. The system of claim 1, wherein the therapeutic agent layer is concentrated in the proximity of a portion of the balloon that becomes an apex portion of the balloon upon inflation.

8. The system of claim 1, wherein the second elastic layer is an oxime cure silicone dispersion.

9. The system of claim 1, wherein the second elongation-at-break percentage is between about 550% and 750%.

10. The system of claim 1, wherein the difference between the first elongation-at-break percentage and the second elongation-at-break percentage is at least 250%.

11. A method of formation of a splittable elastomeric drug delivery device, the method comprising:
forming a first coat on a balloon mandrel;
curing the first coat to form a first elastic layer;
forming a therapeutic agent layer disposed on at least a portion of the first elastic layer;
forming a second coat disposed on the first elastic layer and the therapeutic agent layer; and
curing the second coat to form a second elastic layer,
wherein the first elastic layer has a first elongation-at-break percentage and the second elastic layer has a second elongation-at-break percentage.

12. The method of claim 11, wherein the first elongation-at-break percentage is at least 1000% and the second elongation-at-break percentage is between about 550% and 750%.

13. The method of claim 11, wherein the curing the first coat comprises curing the first coat at an elevated temperature.

14. The method of claim 11, wherein the curing the second coat comprises curing the second coat at room temperature.

15. The method of claim 11, wherein the forming a therapeutic agent layer comprises:
depositing granular particles in at least a portion of the first coat;
exposing the granular particles;
dissolving the granular particles to form expandable pores in at least a portion of the first elastic layer;
inflating the first elastic layer;
depositing at least one therapeutic agent in at least a portion of the expandable pores; and
deflating the balloon.

16. The method of claim 15, wherein the first elastic layer is a platinum cure silicone dispersion.

17. The method of claim 15, wherein the second elastic layer is an oxime cure silicone dispersion.

18. A method for treating a vascular condition, the method comprising:
advancing a splittable elastomeric drug delivery device to a treatment site, the splittable elastomeric drug delivery device comprising a balloon having a first elastic layer, a therapeutic agent layer disposed over at least a portion of the first elastic layer, and a second elastic layer disposed over the first elastic layer and the therapeutic agent layer; and
inflating the balloon,
wherein the inflating splits the second elastic layer to expose the therapeutic agent layer to the treatment site.

19. The method of claim 18, wherein the inflating splits the second elastic layer parallel to the longitudinal axis of the balloon.

20. The method of claim 18, wherein the first elastic layer has a percent elongation-at-break of at least 1000% and the second elastic layer has a percent elongation-at-break between about 550% and 750%.
